(19) United States
(12) Patent Application Publication
(10) Pub. No.: US 2024/0366550 A1
(43) Pub. Date: Nov. 7, 2024

(54) Drug for Treating Prostatitis or Benign Prostatic Hyperplasia (BPH)

(71) Applicant: Beijing University of Chemical Technology, Beijing (CN)

(72) Inventors: Qipeng YUAN, Beijing (CN); Li CHENG, Beijing (CN); Pengtao LIU, Beijing (CN)

(21) Appl. No.: 18/288,799

(22) PCT Filed: Sep. 8, 2022
(86) PCT No.: PCT/CN2022/117848

§ 371 Date: Oct. 27, 2023

(30) Foreign Application Priority Data
Sep. 8, 2021 (CN) 202111047970.3

(51) Int. Cl. A61K 31/26; A61K 9/20; A61K 36/31; A61K 45/06; A61P 13/08

(52) U.S. Cl. CPC A61K 31/26; A61K 9/2054; A61K 9/2059; A61K 36/31; A61K 45/06; A61P 13/08; A61K 2236/15; A61K 2236/331; A61K 2236/37; A61K 2236/51; A61K 2236/55

(57) ABSTRACT

The present disclosure provides a drug for treating prostatitis or benign prostatic hyperplasia (BPH), including sulforaphane (SFA) or an SFA-containing plant extract. In the present disclosure, the SFA, as an active ingredient, is isolated from a plant of Brassica. The plant of Brassica includes, but is not limited to, Raphanus sativus L., Brassica oleracea L. var. italica Plenck, Brassica oleracea, and Brassica juncea (L.) Czern.

### TECHNICAL FIELD

The present disclosure relates to a drug for treating prostatitis or benign prostatic hyperplasia (BPH).

### BACKGROUND

Chronic prostatitis is a common disease that occurs in young and middle-aged men. Chronic prostatitis patients account for about 25% in urology clinics, and about 50% of men with the chronic prostatitis have symptoms in their lifetime. The chronic prostatitis seriously affects the patient's quality of life to a degree comparable to that of myocardial infarction, unstable angina, or active Crohn's disease. Due to the complex and diverse etiology, pathological changes, and clinical symptoms, there is high difficulty in management of the chronic prostatitis among prostate diseases.

Prostatitis is clinically divided into acute prostatitis, chronic prostatitis, and asymptomatic prostatitis. The chronic prostatitis clinically includes chronic bacterial prostatitis and chronic non-bacterial prostatitis/chronic pelvic pain syndrome. For a long time, the drugs for treating prostatitis have been slow to take effect, and can only play some local anti-inflammatory effects during the treatment. This shows no substantial significance for the elimination of inflammation in the entire prostate gland.

A conventional method of drug treatment for chronic prostatitis is to take oral antibiotics for 2 to 4 weeks, and then decide whether to continue antibiotic treatment based on feedback on its efficacy. It is recommended to improve urination symptoms and pain by using u-receptor blockers; plant preparations, non-steroidal anti-inflammatory analgesics, and M-receptor blockers can also be used to relieve the urination symptoms and pain. However, due to the long healing period of chronic prostatitis, long-term use of the above drugs may cause greater side effects. At present, it is recommended to treat prostatitis with traditional Chinese medicines in accordance with relevant standards of the Society of Traditional Chinese Medicine or the Society of Integrative Traditional Chinese and Western Medicine to achieve a desirable curative effect.

As early as 2000, the World Health Organization (WHO) clearly stated that due to a special physiological structure of the prostate, the problem of drug penetration has not been solved. The prostate is surrounded by an extremely dense lipid envelope of the prostate, but inflammations of the prostate occur inside. Ordinary drugs cannot penetrate this dense envelope at all, such that drug ingredients can only move around the prostate glands and eliminate some surrounding inflammations. This is the main reason why prostatitis treatment drugs can only relieve symptoms but cannot cure prostatitis. Accordingly, it has always been a new problem to be solved urgently to develop a drug that can penetrate into the prostate and prostatic fluid and then exert its antibacterial and anti-inflammatory effects. Sulforaphane (SFA) is just a kind of small-molecular natural product with hydrophilic and lipophilic properties. Tested by the method in this application, the oral bioavailability of SFA can reach 90%, indicating that the SFA can quickly penetrate cell membranes in vivo. Afterwards, by testing a concentration of SFA in the patient's prostate fluid obtained by massage, it is verified that the SFA can indeed quickly reach the parts that other drugs cannot do.

### SUMMARY

An objective of the present disclosure is to provide a drug for treating prostatitis or benign prostatic hyperplasia (BPH). The drug can be quickly delivered to a lesion site, thereby effectively treating the prostatitis or BPH without easy relapse after cure.

In the present disclosure, the SFA, as an active ingredient, is isolated from a plant of Brassica. The plant of Brassica includes (but is not limited to) Raphanus sativus L., Brassica oleracea L. var. italica Plenck, Brassica oleracea, Brassica juncea (L.) Czern., and Armoracia rusticana G. Gaertn., B. Mey. & Scherb. This type of active ingredient has a clear therapeutic effect on the chronic prostatitis mentioned above.

The present disclosure further provides a preparation method of a drug for treating prostatitis or BPH, where the drug includes SFA.

Another objective of the present disclosure is to provide use of the SFA in preparation of a drug for treating chronic bacterial prostatitis. The chronic bacterial prostatitis is chronic bacterial prostatitis accompanied by bacterial infection in semen. The bacterial infection is caused by one or more of Staphylococcus epidermidis, Staphylococcus aureus, Escherichia coli, Streptococcus agalactiae, Streptococcus faecalis, Streptococcus viridans, Enterococcus faecalis, and Staphylococcus hemolyticus. The chronic bacterial prostatitis is chronic bacterial prostatitis accompanied by pains and discomfort symptoms as well as urination symptoms. The pains and discomfort symptoms include one or more of distending pain and discomfort in the suprapubic region, perineum pain, penis pain, and ejaculation pain. The urination symptoms include one or more of frequent urination, urgent urination, feeling of incomplete urination, hesitation in urination, labored urination, and white urine. The present disclosure further provides use of the SFA in preparation of a drug for treating chronic non-bacterial prostatitis, and use of the SFA in preparation of a drug for treating BPH.

In the present disclosure, the SFA in treating chronic prostatitis can also be used in food, food additives, and health care products.

In the present disclosure, a main point is the use of the SFA in preparation of a drug for treating chronic bacterial prostatitis. A pharmacological principle is: after continuous administration of SFA, the SFA can enter the interior of the prostate through the extremely dense lipid envelope of the prostate, and then penetrate into the prostatic fluid and semen to reach a certain composition concentration, thereby exerting its antibacterial and anti-inflammatory effects.

In the present disclosure, the use of the SFA in preparation of a drug for treating chronic bacterial prostatitis has the advantages of new uses, exact curative effect, less adverse reactions, and desirable patient compliance with medication compared with those in the existing technology. The SFA can be widely used in chemical composition applications.

The present disclosure is described in detail below with reference to the embodiments.

### DETAILED DESCRIPTION OF THE EMBODIMENTS

The following examples will help to understand the present disclosure, but these examples are only for illustrating the present disclosure, and the present disclosure is not limited to these contents.

The present disclosure provides use of SFA in preparation of a drug for treating chronic bacterial prostatitis, where the SFA is used in preparation of the drug for treating chronic bacterial prostatitis. The chronic bacterial prostatitis is chronic bacterial prostatitis accompanied by bacterial infection in semen. The bacterial infection is caused by one or more of Staphylococcus epidermidis, Staphylococcus aureus, Escherichia coli, Streptococcus agalactiae, Streptococcus faecalis, Streptococcus viridans, Enterococcus faecalis, and Staphylococcus hemolyticus. The chronic bacterial prostatitis is chronic bacterial prostatitis accompanied by pains and discomfort symptoms as well as urination symptoms. The pains and discomfort symptoms include one or more of distending pain and discomfort in the suprapubic region, perineum pain, penis pain, and ejaculation pain. The urination symptoms include one or more of frequent urination, urgent urination, feeling of incomplete urination, hesitation in urination, labored urination, and white urine.

### Example 1

### Drug Efficacy Test

### 1. Materials and Methods

1.1. General information: this group of patients included 117 cases, aged 18 to 52 years old, with an average age of 34.5 years, and their disease duration ranged from 2 months to 5 years. 55 of the males had prostatic hyperplasia, their disease duration ranged from 1 to 5 years, and their main clinical manifestations were:
(1) different degrees of frequent urination, urgent urination, and incomplete urination, as well as urination symptoms such as urination hesitancy, strenuous urination, and white urine;
(2) pains and discomfort symptoms including distending pain and discomfort in the suprapubic region, perineum pain, penis pain, and ejaculation pain; and
(3) routine urine examination before treatment was normal, white blood cells were found to be more than 10 cells/HP by microscopic examination of prostate fluid obtained by massage, and positive bacterial culture of VB3 and EPS was found by Meares-stamey four-cup test, and the patients were confirmed as type-II prostatitis patients and included in this study.

1.2. Treatment method: 10 mg of SFA per day for 4 to 6 weeks. During the treatment period, patients should avoid stimulating diet, took a hot water hip bath, had a regular sexual life, and maintained a well mental state.

1.3. Criteria for determining efficacy: the patients were scored according to the Chronic Prostatitis Score (NIH2CPSI) of the National Institutes of Health (NIH); the efficacy was determined based on the patient's symptoms, routine examination results of prostate fluid obtained by massage, and VB3 and EPS bacterial culture results obtained by the Meares-Stamey four-cup test.

Cure: symptoms and signs disappeared, CPSI score reduced by >90%, symptom-free for >4 weeks, EPS WBC 15 points, EPS WBC <25%, and Meares-Stamey four-cup test showed that VB3 and EPS bacterial culture were positive.

### 2. Results

A total of 115 patients (2 patients were lost to follow-up) completed treatment. As shown in the changes of NIH-CPSI before and after treatment (Table 1), the total score, pain and discomfort score, urination symptom score, and quality of life score were all significantly reduced (P<0.01). Most patients' symptoms were relieved significantly after treatment. 20 cases were cured within 4 weeks of taking the drug and then stopped; while the remaining 95 cases persisted in taking the drug for 6 weeks, of which 15 cases were cured, 44 cases were markedly effective, 23 cases were effective, and 13 cases were ineffective, indicating a total effective rate of 88.7%. Moreover, patients generally had few adverse reactions, and only a few patients had mild intestinal reactions.

TABLE 1
Changes in NIH-CPSI scores before and after treatment

| | | Pain and | Urination | Quality |
|---|---|---|---|---|
| | NIH-CPSI | discomfort | symptom | of life |
| Time | score | score | score | score |
| Before treatment | 35.2 ± 3.3 | 15.3 ± 2.6 | 6.8 ± 1.1 | 15.2 ± 4.6 |
| After treatment | 20.1 ± 5.6 | 10.3 ± 3.2 | 3.2 ± 1.1 | 7.5 ± 1.3 |
| P value | <0.01 | <0.01 | <0.01 | <0.01 |

In addition, through further statistics, it was found that sulforaphene had a more obvious therapeutic and alleviating effect on urination symptoms (frequent urination, urgent urination, and white urine) and pains and discomfort symptoms (distending pain and discomfort in the suprapubic region and penis pain) of chronic bacterial prostatitis. The relieving rate of different symptoms (calculated by the number of patients who were relieved/the total number of patients) was shown in Table 2:

| | | | Feeling of | | |
|---|---|---|---|---|---|
| Urination | Frequent | Urgent | incomplete | Labored | White |
| symptoms | urination | urination | urination | urination | urine |
| Relieving | 80.2% | 81.3% | 67.1% | 72.6% | 91.2% |
| rate | | | | | |

| | Distending pain | | | |
|---|---|---|---|---|
| Pains and | and discomfort in | | | |
| discomfort | the suprapubic | Perineum | Penis | Ejaculation |
| symptoms | region | pain | pain | pain |
| Relieving rate | 82.9% | 69.5% | 77.3% | 81.2% |

TABLE 3
Routine examination results of prostatic fluid
obtained by massage before and after treatment

| | Before treatment (number of people) | | | | | After treatment (number of people) | | | | |
|---|---|---|---|---|---|---|---|---|---|---|
| Item | Negative | + | ++ | +++ | ++++ | Negative | + | ++ | +++ | ++++ |
| White | 0 | 31 | 44 | 28 | 14 | 32 | 49 | 21 | 9 | 4 |
| blood cells | | | | | | | | | | |

TABLE 4
Distribution of pathogenic bacteria in prostatic
fluid obtained by massage before treatment

| Bacterial name | Number of plants | Proportion % |
|---|---|---|
| Staphylococcus epidermidis | 59 | 50.43 |
| Staphylococcus aureus | 15 | 12.82 |
| Escherichia coli | 12 | 10.26 |
| Streptococcus agalactiae | 11 | 9.4 |
| Streptococcus faecalis | 8 | 6.84 |
| Streptococcus viridans | 5 | 4.27 |
| Enterococcus faecalis | 4 | 3.42 |
| Staphylococcus hemolyticus | 3 | 2.56 |

The prostatic fluid obtained by massage from 115 patients was subjected to bacterial culture, and a total of 117 strains of bacteria were isolated (including 2 strains of bacteria isolated from 2 patients). The strains and their proportions were shown in Table 4. According to the statistics of the therapeutic effect after treatment, 12 patients were infected with Escherichia coli, 11 patients were infected with Streptococcus agalactiae, and 8 patients were infected with Streptococcus faecalis, the total effective rate reached 10000.

The drug concentration of SFA in vivo was determined in 9 cases of the 115 patients: the patients took 10 mg of SFA orally every day for 3 consecutive days. The determination was conducted 6 h after taking the drug on the 3rd day, and the results were shown in Table 5:

TABLE 5
Concentrations of sulforaphene in different tissue
fluids after oral administration with sulforaphene

| Site | Concentration (ng/mL) |
|---|---|
| Blood | 50.6-182.3 |
| Semen | 2.6-5.5 |
| Prostatic fluid obtained by massage | 13.5-22.8 |

TABLE 6
Examination results of male prostatic
hyperplasia before and after treatment

| | Prostatic hyperplasia | | | |
|---|---|---|---|---|
| Item | Cured | Effective | Ineffective | Total |
| Number of subjects | 52 | 2 | 1 | 55 |

### Example 2

100 kg of broccoli seeds were crushed or homogenized, 2,000 L of 20 times a volume of deionized water was added, stirred at room temperature to allow hydrolysis 5 h, hydrochloric acid was added to adjust a pH value of a resulting hydrolyzate to 2.0, allowed to stand overnight, and centrifugation was conducted to obtain 2,000 L of an SFA hydrolyzate as a supernatant; (2) the sulforaphene hydrolyzate obtained in step (1) was extracted 3 times with 6,000 L of ethyl acetate (2,000 L of the ethyl acetate each time), 6,000 L in total of an extraction layer in the ethyl acetate was collected, the extraction layer was distilled under reduced pressure at 40° C. and a vacuum degree of −0.08 MPa to obtain 200 L of a crude extract of the SFA, while the ethyl acetate was recovered; (3) the crude extract of SFA obtained in step (2) were added to a molecular distillation device to allow first-stage molecular distillation to remove residual ethyl acetate, moisture, and low-boiling point impurities in the crude extract of SFA, a heavy component flowing out from a distillation wall of a first-stage molecular distillation device was collected to obtain 2,000 g of a first-stage molecular distillation heavy component; where the conditions for the first-stage molecular distillation included: raw material insulation temperature of 60° C., vacuum degree of 2,000 Pa, distillation temperature of 100° C., feeding flow rate of 2 mL/min, condensation surface temperature of 0° C., and film scraper speed of 400 rpm; and (4) the first-stage molecular distillation heavy component obtained in step (3) was added into the molecular distillation device to allow second-stage molecular distillation to remove high-boiling point impurities in the first-stage molecular distillation heavy component, a light component flowing out from a condensation surface of a second-stage molecular distillation device was collected to obtain 1,000 g of the SFA with a high purity of 98%; where the conditions for the second-stage molecular distillation included: raw material insulation temperature of 70° C., vacuum degree of 0.1 Pa, distillation temperature of 110° C., feeding flow rate of 1 mL/min, condensation surface temperature of 0° C., and film scraper speed of 450 rpm.

### Example 3

100 g of the SFA extract prepared by the above method and 19.9 kg of dextrin were mixed and dissolved in 100 L of deionized water, and then spray-dried. The spray drying was conducted at an air inlet temperature of 180° C., an air outlet temperature of 80° C., and a liquid inlet speed of 5 L/h. A product was collected to prepare a spray-dried powder of SFA to allow granulation in a granulator, and obtained granules were sub-packed in 2 g by an automatic granule packaging machine to prepare the SFA water-soluble granule with a 0.5% SFA content (by mass fraction, the percentages not otherwise stated below were all mass fractions).

### Example 4

100 g of the SFA extract prepared by the above method and 9.9 kg of dextrin were mixed and dissolved in 50 L of deionized water, and then spray-dried in a same manner as that in Example 1 to prepare a spray-dried powder of SFA. The powder was sub-packed in 1 g to prepare the SFA water-soluble granule with a 1% SFA content.

### Example 5

100 g of the SFA extract prepared by the above method and 1.9 kg of dextrin were mixed and dissolved in 10 L of deionized water, and then spray-dried in a same manner as that in Example 1 to prepare a spray-dried powder of SFA with a 5% SFA content. 50 g of magnesium stearate, 500 g of Peng Su Wang (super carboxymethyl starch sodium), and 2.45 kg of microcrystalline cellulose were added to the dried powder, mixed well to allow dry granulation, and then obtained drug-containing granules were tableted to obtain the oral tablet containing 2% SFA with a specification of 500 mg per tablet.

### Example 6

200 g of the SFA extract prepared by the above method and 1.8 kg of dextrin were mixed and dissolved in 10 L of deionized water, and then spray-dried in a same manner as that in Example 1 to prepare a spray-dried powder of SFA with a 10% SFA content. 50 g of magnesium stearate, 500 g of Peng Su Wang, and 2.45 kg of microcrystalline cellulose were added and mixed well to allow tableting to obtain the oral tablet containing 4% SFA with a specification of 500 mg per tablet.

### Example 7

100 g of the SFA extract prepared by the above method and 0.9 kg of dextrin were mixed and dissolved in 5 L of deionized water, and then spray-dried in a same manner as that in Example 1 to prepare a spray-dried powder of SFA with a 10% SFA content. 100 g of magnesium stearate, 1 kg of Peng Su Wang, and 7.9 kg of microcrystalline cellulose were added and mixed well to allow tableting to obtain the oral tablet containing 1% SFA with a specification of 500 mg per tablet.

1. A drug for treating prostatitis or benign prostatic hyperplasia (BPH), comprising sulforaphane (SFA) or an SFA-containing plant extract.

2. The drug according to claim 1, wherein the drug is administered by injection or oral administration.

3. The drug according to claim 1, wherein the SFA is administrated at a dose of 1 mg/person to 150 mg/person daily for 1 week to 24 weeks.

4. The drug according to claim 1, wherein the plant extract is extracted from a plant selected from the group consisting of Brassica oleracea var. gemmifera Zenker, Brassica oleracea var. botrytis Linnaeus, Brassica rapa var. glabra Regel, kale, Brassica oleracea var. acephala DC., broccoli sprouts, Brassica oleracea var. albiflora Kuntze, cauliflower, Brassica juncea var. napiformis Pailleux et Bois, mustard, Brassica rapa L., Raphanus sativus L., Eruca vesicaria subsp. sativa (Miller) Thellung, and Nasturtium officinale R. Br. ex W. T. Aiton.

5. The drug according to claim 1, wherein the SFA or the SFA-containing plant extract is used alone; alternatively, the SFA and the SFA-containing plant extract are used simultaneously, or prepared into a composition with an auxiliary material for use.

6. The drug according to claim 5, wherein the composition is in the form of a drug, a health care product, a food, or a cosmetic.

7. A method for treating prostatitis or BPH using SFA or an SFA-containing plant extract.

8. The method according to claim 7, wherein the SFA or the SFA-containing plant extract is used alone or in combination with a drug.

9. The method according to claim 8, wherein the use in combination with a drug comprises: use in combination with one or more western medicines, use in combination with a Chinese herbal medicine, use in combination with radiotherapy, use in combination with gene therapy, or use in combination with a biological regulator.
